(19) United States
(12) Patent Application Publication
(10) Pub. No.: US 2003/0194423 A1
(43) Pub. Date: Oct. 16, 2003

(54) Composition for enhancing nutritional content of food

(75) Inventors: Allan A. Torney, Brampton (CA); Liisa Mooney, Toronto (CA); Peter Slusarczyk, Fergus (CA)

Correspondence Address: FULBRIGHT & JAWORSKI, LLP, 1301 MCKINNEY, SUITE 5100, HOUSTON, TX, 77010-3095, US

(73) Assignee: MARS, INC., McLean, VA

(21) Appl. No.: 10/122,832

(22) Filed: Apr. 15, 2002

(51) Int. Cl. A61K 31/70; A61K 47/00; A23K 1/165; A23K 1/17

(52) U.S. Cl. 424/442; 426/635; 514/23

(57) ABSTRACT

The present invention is directed to a ready-to-use composition for supplementing nutritional content of a pet food. The composition comprises, on a dry matter basis, from about 15 to about 80% by weight of a protein component, from about 20 to about 85% by weight of a humectant, and from about 1 to about 50% by weight of a lipid component. The composition does not require sterilization or addition of chemical preservative, thereby making the invention suitable for delivery of nutritional functional ingredients that are heat labile.

### BACKGROUND OF THE INVENTION

1. Field of the Invention

The present invention generally relates to a ready-to-use composition for delivering nutrients and supplementing a food product. More particularly, the composition includes completely digestible ingredients that formulate a microbiologically stable composition that is readily added to a food product, preferably as a food coating or mixed in a drink. Processes of preparation and administration are also contemplated.

2. Related Art

Individual animals vary greatly in their nutritional needs depending upon such factors as age, weight, sex and the extent of their exercise or work. Young animals may require twice the nutrients of adult animals. For example, puppies increase their whelping weight by approximately 60 times during their first year of life. In order to do this, they require especially high amounts of protein and vitamins. Older animals have an increased demand for vitamins and minerals in order to maintain the proper function of their bodily systems. Animals in gestation may require up to 20% more nutrients than their own maintenance requirements, and after parturition, the lactating animal may require two to three times her maintenance intake in order to produce the necessary colostrum and milk to nourish the newborn young.

It is recognized that the environment a companion animal is living in greatly affects its nutritional requirements. For example, a cold-acclimated dog has an increased oxygen demand and metabolic rate, and may require 70%-90% more calories than a dog in a milder climate or a dog maintained indoors. Working dogs also have increased oxygen consumption rates that may be as much as eight times greater than a dog at rest, thereby requiring as much as four times as many calories per day as would otherwise be required for maintenance in order to produce the necessary energy to complete the work. If a working dog does not receive the proper nutritional needs, it may begin to suffer within as short a time period as an hour or two from hypoglycemia and in more severe conditions even convulsions and ataxia.

Additionally, nutritionally complete diet compositions have been increasingly employed over the past decade for nutritional support of undernourished patients or patients with gastrointestinal pathology. These dietary compositions are typically marketed as a dry blended or a spray-dried powder that is reconstituted with water prior to consumption. The reconstituted liquid elemental diet is usually fed within two hours in most cases and generally should not be held for more than 24 hours, even when stored at refrigerated temperatures. Similarly, a dry powder nutritional supplement for pets with special needs are available from Purina® Petcare (Health Packs™) that are applied directly to the pet food, and the powder contains vitamins and minerals and other nutritional supplements targeted at specific body systems (i.e., Skin & Coat, Immune Support).

Although improvements in these compositions have evolved with the increasing widespread use of these diets in nutritional management, problems have persisted. Previous liquid diet compositions exhibit unacceptable levels of deterioration when subjected to the heat treatment required for effective sterilization, or even under the more ordinary conditions and ambient temperatures associated with shelf life studies. In addition to bacterial contamination, two other major underlying causes responsible for physical and nutritional deterioration of prior liquid diet compositions are recognized. One type of deterioration, primarily physical, results from emulsion breakdown of the liquid composition which results in the oiling out of the lipid phase, or from precipitation of a solid, usually a mineral compound. This is especially problematic in elemental diets that are frequently administered by enteral tube feeding via nasogastric tubing and such. In this situation, the physical stability of the liquid as a single phase is a critical prerequisite. A second type of deterioration is due to the well known chemical reaction (i.e. browning) between an amine groups of an amino acid moiety and a carbonyl group of a carbohydrate or functionally equivalent chemical moieties. Processing may also result in loss of nutritional quality.

For this reason, ready-to-use liquid elemental diets have not been practical because they do not withstand the rigors of processing, such as sterilization, or maintain stability during the periods of storage required for such manufactured items. The deterioration process is accelerated by increased temperatures, which is an integral part of processing of most food compositions.

Pet food compositions are also subject to deterioration in transit, although deterioration is not usually a problem as long as the finished product is not shipped over long distances. As a result, manufacturers face the choice of exporting finished product over long distances from existing full capacity plants, or investing in full capacity plants in newly opened markets. Manufacturers who choose to export finished product over long distances face a risk of product loss due to deterioration during shipping, handling and storage.

U.S. Pat. No. 4,497,800 to Larson et al. teaches a stable liquid diet composition that, because of an acidic emulsion, maintains its integrity during sterilization. U.S. Pat. No. 4,414,238 to Schmidl teaches a liquid elemental diet comprising, based on total calories, 50 to 90% of a carbohydrate, 5 to 30% of an amino acid component, 10 to 50% of a lipid component, wherein the pH of the composition is in a critical range of 3.0 to 4.4 and thus, yields a stable, heat-sterilizable liquid. U.S. Pat. No. 4,070,488 to Davis teaches a stable aqueous solution useful as a nutritive supplement comprising water, ascorbic acid, iron in a critical amount and gelatin, which retards deterioration of ascorbic acid in the presence of iron but is present in an amount that is non-gelling. U.S. Pat. No. 5,017,389 to Green describes a neutral nutritional drink (e.g., 90 to 98% water) for dogs comprising carbohydrates, electrolytes and vitamin and mineral supplements.

However, all of these prior art liquid nutritional supplements require sterilization or pasteurization prior to use, thereby increasing the rate of deterioration of heat labile ingredients.

A major objective, therefore, of the present invention is to provide a composition in ready-to-use form which possesses adequate and microbiological stability compared with previous diet formulations, so that physical and nutritional quality is not lost during sterilization processes required in the prior art. In contrast to the varying nutritional needs of animals as described above, pet food is typically designed to meet the nutritional requirements of an average animal. Of course, an individual animal's nutritional needs may vary widely from that of the average animal. Thus, a need clearly exists for a product that supplements the nutrition provided by pet food. Preferably, this product should be healthy, palatable and easy for the owner to use.

A second objective of the present invention is to provide a ready-to-use composition useful for delivery of nutrients, in particular nutrients required by both humans and companion animals, that are above or below average in weight and overall health. To improve nutritional performance in a diet composition, the present invention comprises a novel delivery composition that is microbiologically stable and does not require sterilization or pasteurization. Protein quality, somewhat difficult to assess, is a major determinant of nutritional performance in these diets. Measured in nutritional studies, retention of dietary nitrogen by a patient being fed a liquid diet is a key indicator of the diet's protein quality.

Pet food compositions specially formulated to prevent or treat obesity in animals have been described. U.S. Pat. No. 6,071,544 teaches that a specific combination of long chain conjugated fatty acids (0.2 to 1.5 weight % of dry matter), together with up to 50% protein, promotes weight loss in cats. A diet greater than about 16% by weight of animal-based protein is taught to reduce body fat in geriatric dogs (WO 00/51443). However, U.S. Pat. No. 5,141,755 teaches that non-meat based animal fats in a high protein, high farinaceous diet comprises ovo or lacto-ovo in a nutritionally balanced pet food product. U.S. Pat. No. 4,892,748 describes a low calorie dog treat that is comprised primarily of cellulose, a β-1,4-glucan that affords no nutritional value to the animal.

Natural products derived from plants and food sources have frequently provided a rich source of effective compounds, and in recent years there has been an increased interest in the potential nutritional and therapeutic benefit of these natural products. For this reason, functional ingredients are often added to pet foods in order to effect a particular and desired metabolic response. For example, EP 646325A1 describes a pet food comprised of at least 30% by weight of indigestible dextrin that demonstrates obese-improving effects by controlling blood-sugar levels and insulin secretion in dogs and cats. U.S. Pat. No. 5,962,043 teaches jojoba seed meal as a nutritional supplement in animal feed, especially companion dogs, to promote weight loss. Simmondsin is described as the active or functional ingredient, that based on previous research, results in an associated reduction in food intake and retardation of growth. These formulations include high fat and high caloric density to ensure palatability.

U.S. Pat. No. 6,204,291 teaches dietary supplementation with L-carnitine, a naturally occurring acid also known as β-Hydroxy-γ-trimethylaminobutyrate, to dog food to promote weight loss. Carnitine is found in the body and is enzymatically combined with fatty acids to facilitate their transportation through mitochondrial membranes, thus aiding in fatty acid metabolism (Yalkowsky, S. H., 1970). Oral administration of L-carnitine for obesity in mammals has been described in U.S. Pat. No. 3,810,994. It also has been implicated in improvements in myocardial contractility and systolic rhythm in congestive heart failure, it has been administered in cases of cardiac arrythmia (U.S. Pat. No. 3,830,931; and U.S. Pat. No.3,968,241), and it has been used for increasing the level of high density lipoproteins (U.S. Pat. No. 4,255,449).

Prior to the present invention, current solutions have not provided a ready-to-use composition that does not require heat sterilization, and thus, the prior art compositions lack ingredients that are heat labile. The ready-to-use composition of the present invention represents a compositional delivery system for nutrients which is formulated and microbiologically stable to yield a liquid, ready-to-use form that is superior physical stability and nutritional utilization as compared to reconstituted diet formulations of the prior art.

### SUMMARY OF THE INVENTION

The present invention provides a ready-to-use composition for supplementing the nutritional content of pet food comprising, on a dry matter basis, from about 15 to about 80% by weight of a protein component, from about 20 to about 85% by weight of a humectant, and from about 1 to about 50% by weight of a lipid component, wherein the composition is microbiologically stable. In further embodiments, the inventive composition further comprises from about 0.1 to about 49% by weight of a functional ingredient. Functional ingredients that are in heat labile such as an essential oil, a volatile molecule having a fragrance, a molecule that is readily oxidized, an enzyme, an antibiotic, or a probiotic are contemplated. Heat-labile ingredients include, but are not limited to, green lipped mussel powder, cololstrum, and lactobaccili.

In certain embodiments, the protein component comprises hydrolyzed proteins that are obtained from an animal or a plant source. Non-limiting examples include beef, swine, sheep, fish, or poultry, and non-limiting examples of the plant source include wheat, an alfalfa or a legume source. In specific embodiments, the protein component comprises gelatin.

In certain embodiments, the humectant comprises a sugar or a polyhydroxyl alcohol or a mixture of a sugar and a polyhydroxyl alcohol. The mixtures contemplated comprise the sugar and the polyhydroxyl alcohol in a ratio of about 1:1, in a ratio of about 1:2, or in a ratio of about 2:3.

In specific embodiments, the lipid component is a fatty acid such as lauric acid, myristic acid, palmitic acid, stearic acid, arachidic acid, behenic acid, lignoceric acid, palmitoleic acid, oleic acid, linoleic acid, alpha-linoleic acid, gamma-linoleic acid, arachidonic acid, nervonic acid, eicosapentanoic acid or derivatives thereof including glycerol esters and alkyl esters, or fatty acids having at least one unsaturation, glycosylation or alkylation or the like. The lipid component is preferably provided as a vegetable oil such as coconut oil, corn oil, cotton seed oil, olive oil, safflower oil, sunflower oil, soybean oil, or an equivalent oil.

In further embodiments, the inventive composition further comprises a vitamin or a mineral.

In certain embodiments, the inventive composition is an aqueous solution, a liquid concentrate, or a colloidal suspension, and is characterized by having a physical consistency of a gel, a sol-gel, a gravy, or a syrup.

It is an preferred that the inventive composition have a pH in the range of 4.0 to 8.0, or more preferably, a pH in the range of about 5.0 to about 6.5, and most preferably, a pH in the range of about 5.5 to about 6.5.

It is an object of the present invention to provide a process for producing a ready-to-use composition for supplementing the nutritional content of a pet food comprising the step of mixing, on a dry matter basis, from about 15 to about 80% by weight of a protein component, from about 20 to about 85% by weight of a humectant, and from about 1 to about 50% by weight of a lipid component to form the composition, wherein the composition is microbiologically stable.

In a further embodiment, the composition is adsorbing on the outer surface of the pet food, such as a food topper. In yet further embodiments, from about 0.1 to about 49% by weight of a functional ingredient is added to the composition, wherein the functional ingredient is heat labile or not heat labile. In certain embodiments, the functional, heat labile ingredient is an essential oil, a volatile molecule having a fragrance, a molecule that is readily oxidized, denatured or inactivated, an enzyme, an antibiotic, or a probiotic.

In other embodiments, the composition further comprises a vitamin or a mineral. In specific embodiments, the protein component comprises hydrolyzed proteins, that are obtained from an animal or a plant source. In preferred embodiments, the protein component comprises gelatin.

In specific embodiments, the humectant comprises a sugar, a polyhydroxyl alcohol or a mixture of a sugar and a polyhydroxyl alcohol in a ratio of about 1:1, in a ratio of about 1:2, or in a ratio of about 2:3.

In specific embodiments, the lipid component is a fatty acid or a derivative thereof, such as a glycerol ester or an alkyl ester. In other specific embodiments, the lipid component is provided as a vegetable oil including coconut oil, corn oil, cotton seed oil, olive oil, safflower oil, sunflower oil, soybean oil, or an equivalent oil.

In certain embodiments, the composition is an aqueous solution, a liquid concentrate, or a colloidal suspension. In other specific embodiments, the composition is characterized by having a physical consistency of a gel, a sol-gel, a gravy, or a syrup.

It is preferred that the composition have a pH in the range of 4.0 to 8.0, in the range of about 5.0 to about 6.5, or in the range of about 5.5 to about 6.5.

It is another object of the present invention to provide a process for producing a ready-to-use composition for supplementing the nutritional content of a pet food, the process comprising the steps of adding from about 15 to about 80% by weight of a protein component to an equal amount of water to form a solution; mixing into said solution from about 20 to about 85% by weight of a carbohydrate component to form a mixture; and combining from about 1 to about 50% by weight of a lipid component to the mixture to produce the composition.

In further embodiments, from about 0.1 to about 49% by weight of a functional ingredient is added to the mixture or the composition. In certain embodiments, the functional ingredient is heat labile. In certain embodiments, the functional, heat labile ingredient is an essential oil, a volatile molecule having a fragrance, a molecule that is readily oxidized, denatured or inactivated, an enzyme, an antibiotic, or a probiotic.

In yet further embodiments, the process further comprises adding a vitamin or a mineral to the composition.

In specific embodiments, the protein component comprises hydrolyzed proteins, such as those obtained from an animal or a plant source. In a specific embodiment, the protein component comprises gelatin.

In certain embodiments, the humectant comprises a sugar or a polyhydroxyl alcohol.

In other specific embodiments, the humectant comprises a mixture of a sugar and a polyhydroxyl alcohol. In further specific embodiments, the mixture comprises the sugar and the polyhydroxyl alcohol in a ratio of about 1:1, in a ratio of about 1:2, or in a ratio of about 2:3.

In specific embodiments, the lipid component is a fatty acid or a derivative thereof such as a glycerol ester or an alkyl ester.

In other specific embodiments, the composition is an aqueous solution, a liquid concentrate, or a colloidal suspension. In yet other specific embodiments, the composition is characterized by having a physical consistency of a gel, a sol-gel, a gravy, or a syrup.

It is preferred that the composition has a pH in the range of 4.0 to 8.0, or preferably in the range of about 5.5 to about 6.5.

An additional object of the present invention is a process for delivering a nutrient to an animal comprising the step of feeding to an animal a ready-to-use composition comprising, on a dry matter basis, from about 15 to about 80% by weight of a protein component, from about 20 to about 85% by weight of a humectant, from about 1 to about 50% by weight of a lipid component, and from about 0.1 to 49% of an essential nutrient to form a nutritional delivery composition, wherein the composition is microbiologically stable.

A further object of the present invention is a process of promoting nutrition in a companion animal comprising the step of feeding to said animal a pet food having an outer layer comprised of from about 15 to about 80% by weight of a protein component, from about 20 to about 85% by weight of a humectant, and from about 1 to about 50% by weight of a lipid component, wherein the composition is microbiologically stable.

In further embodiments, the composition further comprises about 0.1 to about 49% by weight of a functional ingredient, and the functional ingredient can be heat labile. In certain embodiments, the functional, heat labile ingredient is an essential oil, a volatile molecule having a fragrance, a molecule that is readily oxidized, denatured or inactivated, an enzyme, an antibiotic, or a probiotic.

In specific embodiments, the protein component comprises hydrolyzed protein, such as gelatin. In other specific embodiments, the humectant comprises a mixture of a sugar and a polyhydroxyl alcohol. In an additional specific embodiment, the composition has a pH in the range of from about 5.5 to about 6.5.

It is another object of the present invention to provide a ready-to-use composition for delivering a pharmaceutical or a medicament comprising on a dry matter basis, from about 15 to about 80% by weight of a protein component, from about 20 to about 85% by weight of a humectant, from about 1 to about 50% by weight of a lipid component, and a therapeutically effective amount of the pharmaceutical or the medicament, wherein the composition is microbiologically stable.

It is preferred that the pharmaceutical or the medicament is water soluble, which includes a solution comprising the pharmaceutical or medicament that is a suspension, a colloidal suspension, an emulsion or a liposomal complex. In specific embodiments, the pharmaceutical is an antibiotic or a probiotic.

In certain embodiments, the protein component comprises hydrolyzed proteins that are from an animal source or a plant source. In preferred specific embodiments, the protein component comprises gelatin.

In specific embodiments, the humectant comprises a sugar, a polyhydroxyl alcohol or a mixture of a sugar and a polyhydroxyl alcohol. In specific embodiments, the mixture comprises the sugar and the polyhydroxyl alcohol in a ratio of about 1:1, a ratio of about 1:2, or a ratio of about 2:3.

In certain embodiments, the lipid component is a fatty acid or a derivative thereof. The lipid component is, in a specific embodiment, provided as a vegetable oil. The inventive composition is an aqueous solution, a liquid concentrate, or a colloidal suspension and is characterized by having a physical consistency of a gel, a sol-gel, a gravy, or a syrup.

In preferred specific embodiments, the composition has a pH in the range of about 4.0 to about 8.0, or more preferably, a pH in the range of about 5.0 to about 6.5, or most preferably, a pH in the range of about 5.5 to about 6.5.

A further object of the present invention is a process for delivering medicine to an animal comprising the steps of mixing, on a dry matter basis, about 15 to about 80% by weight of a protein component, about 20 to about 85% by weight of a humectant, about 1 to about 50% by weight of a lipid component, to form a delivery composition, wherein the composition is microbiologically stable; adding a therapeutically effective amount of a pharmaceutical in a pharmaceutically acceptable diluent to form a medicinal delivery composition; and feeding to the animal the medicinal delivery composition.

In specific embodiments, the pharmaceutical is an antibiotic or a probiotic. In other specific embodiments, the animal is fed orally or enterally.

In certain embodiments, the protein component comprises gelatin, and the humectant comprises a mixture of a sugar and a polyhydroxyl alcohol. In specific embodiments, the composition has a pH in the range of about 5.5 to about 6.5.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DETAILED DESCRIPTION OF THE INVENTION

### Definitions

The term “functional ingredient” as used herein refers to a compound, naturally occurring or synthetic, that is included in a composition and effects a preventative and/or therapeutic response by modulating metabolism in a manner found to be specific to the compound. “Functional ingredients” can also include pharmaceuticals and/or medicaments.

A compound, component or composition is said to be “acceptable” if its administration can be tolerated by a recipient mammal. Such a component is said to be administered in an “effective amount” if the amount administered is physiologically significant. A component is physiologically significant if its presence results in technical change in the physiology of a recipient mammal. For example, in weight management of companion animals, an agent which slows, hinders, delays, completely treats the disease and/or symptoms of obesity, is considered effective.

The term “therapeutically effective amount” as used herein is defined as the amount of a molecule or a compound required to improve a symptom associated with a disease. For example, in the treatment of cancer such as breast cancer, a molecule or a compound which decreases, prevents, delays or arrests any symptom of the breast cancer is therapeutically effective. A therapeutically effective amount of a molecule or a compound is not required to cure a disease but will provide a treatment for a disease. A molecule or a compound is to be administered in a therapeutically effective amount if the amount administered is physiologically significant. A molecule or a compound is physiologically significant if its presence results in technical change in the physiology of a recipient organism.

### The Present Invention

It is known that carbohydrates are the primary fuel for muscular exercise in man. Proteins and fats are indirect fuels. Protein either supplies amino acids for tissue synthesis or supplies fuel for energy requiring processes during periods of nitrogen acquisition. If carbohydrates are not available in foods, the fuel must be made by the body from those materials which are in the diet. The great demand for fuel accompanying muscular exercise may rapidly exhaust carbohydrate stores evidenced by a decrease in glycogen in liver and muscles. If exercise is sufficiently severe and prolonged, abnormal lowering of the blood-sugar level may result. These phenomena are accompanied by increased breakdown of body protein (excreted as nitrogen in the urine).

Therefore, it is an object of the present invention to provide a composition that is useful to deliver nutrients to a human or a companion animal. The composition is a ready-to-use composition for supplementing the nutritional content of pet food comprising, on a dry matter basis, from about 15 to about 80% by weight of a protein component, from about 20 to about 85% by weight of a humectant, and from about 1 to about 50% by weight of a lipid component, wherein the composition is microbiologically stable.

By “microbiologically stable” is meant that the ready-to-use composition does not require processing, i.e., sterilization or pasteurization.

The inventive composition may further comprise from about 0.1 to about 49% by weight of a functional ingredient. Because the composition does not require processing, functional ingredients that are in heat labile such as an essential oil, a volatile molecule having a fragrance, a molecule that is readily oxidized such as an omega-3 fatty acid, an enzyme, an antibiotic or a probiotic are contemplated. One of ordinary skill in the art recognizes that probiotics comprise living microbes and aid in maintaining a balance of the natural flora of the digestive system.

The protein component as used herein, refers to a protein including peptides, amino acids, protein salts (caseinates), and protein hydrolysates. In preferred embodiments, the protein component is a protein hydrolysate, which comprises a heterologous mixture of water-soluble proteins of high average molecular weight. The protein hydrolysate does not have to be complete in that it provides all amino acids, essential or otherwise. Non-limiting examples of protein hydrolysates include gelatin, lactalbumin, whey protein hydrolysates, and soy hydrolysates.

The protein can be derived from any animal (mammal, cold-blooded aquatic, and poultry) or plant source. Preferably, the protein is water soluble at a pH in the range of 4.0 to 8.0, of 5.0 to 6.5, or of 5.5 to 6.5. The protein can be an isolate from the muscle such as tendons or ligaments or an organ portion of the animal such as the skin, the blood fluid or the lacteal fluid. The protein can also be isolated from poultry eggs. The animal source is, for example, beef, swine, sheep, fish, or poultry. The plant proteins which can be used can be obtained from any grain such as wheat, leaf protein such as alfalfa or legume source.

In preferred embodiments, the use of gelatin in the above composition provides a source of substantially all of the amino acids recognized as being essential to nutrition. Thus, the gelatin not only acts to stabilize the composition, but also serves to provide an inexpensive source for these essential amino acids, which are readily absorbed by the gut without prior digestion.

The humectant ingredient aids in the microbiological stability of the composition. Humectants include, but are not limited to, propylene glycol, and other ingredients to prevent microbial growth (bacteria and mold). In certain embodiments, the humectant comprises a sugar or a polyhydroxyl alcohol or a mixture of a sugar and a polyhydroxyl alcohol. The mixtures contemplated comprise the sugar and the polyhydroxyl alcohol in a ratio of about 1:1, in a ratio of about 1:2, or in a ratio of about 2:3.

Further, the humectant of the present invention also serves as a carbohydrate source. For example, dextrose may be used which is not as sweet as either fructose or glucose and, as such, the composition is quite palatable to substantially all animals without the addition of an artificial flavoring. Simple sugars dissolve easily in water and do not need to be digested before utilization by the animal. Thus, sugars including dextrose (e.g., glucose), fructose, granulated sugar and invert sugar, represent a source of quick energy. Each of these sugars is an energy food fully utilizable by cells of the body, each of the glucose and fructose sugars acting in a particular manner during protracted physical exertion. For example, glucose is easily and rapidly transported out of the digestive system into the blood whereas fructose is more passively and slowly transported. Once into circulation, the fructose is somewhat more efficient insofar as initial transport requires less energy and subsequent utilization for energy proceeds more readily. Thus, both immediate and longer lasting benefits are attainable. This last factor is particularly important where the animal or human is being subjected to high stress levels. Such stress levels are often encountered by working mammals (i.e., working dogs), particularly during excessively hot and humid summer months. These stress levels are also suffered by animals and humans recovering from injuries and/or surgical procedures.

The carbohydrate component of the humectant may be any edible or FDA approved carbohydrate which is digestible. In general, the carbohydrate contribution, as a percent of total calories in the diet, may range from about 20 to about 85%. Non-limiting examples of a carbohydrate source is maltodextrins, a low molecular weight hydrolyzed cornstarch or carbohydrates supplied as organic acid anions, e.g. citrate, gluconate, glycerophosphate.

The lipid component of the inventive composition provides nutritional value and palatability. In specific embodiments, the lipid component is provided as a vegetable oil or as a fatty acid. Non-limiting examples of a suitable vegetable oil include coconut oil, corn oil, cotton seed oil, olive oil, safflower oil, sunflower oil soybean oil, or an equivalent oil. Further, the fatty acid may be provided in various forms. A non-exhaustive list of suitable fatty acids include lauric acid, myristic acid, palmitic acid, stearic acid, arachidic acid, behenic acid, lignoceric acid, palmitoleic acid, oleic acid, linoleic acid, alpha-linoleic acid, gamma-linoleic acid, arachidonic acid, nervonic acid, eicosapentanoic acid or derviatives thereof such as an unsaturation, a glycosylation or an alkylation. In general, a fatty acid molecule comprises a carboxylic acid with long-chain hydrocarbon side groups covalently attached. Natural fat components such as, for example, safflower oil, soybean oil, corn oil, cotton seed oil, coconut oil, olive oil, and the like, may be used. Alternatively, fat may be provided in a chemically defined form, such as the essential fatty acids or their glycerol esters or alkyl esters. One of ordinary skill in the art is aware that an essential fatty acid is a molecule that is not metabolized by the animal or human but is required by the body, and, thus, must be obtained through dietary consumption. For the diet of this invention, it is preferred that from 1 to 50% of the lipid component is provided in the inventive composition. Additional lipids may be present as soy oil or in the form of the fatty acid esters comprising the glyceride stabilizing agent.

From the above, it should be appreciated that the nutritional drink composition of the present invention can include a vitamin and/or mineral supplement, particularly if the composition is also to be utilized to aid in the treatment of various ailments and diseases. These conditions include anorexia, anemias, exposure, hypoglycemia, liver diseases, pancreatitis, renal disease and urolythiasis. Those skilled in the art appreciate that minimum requirements have been established for certain vitamins and minerals that are known to be necessary for normal physiological function. Practitioners also understand that appropriate additional amounts (overages) of vitamin and mineral ingredients need to be provided to compensate for some loss during processing and storage of such diets. Therefore, it is contemplated that a vitamin can be added to the inventive composition, such as including but are not limited to, vitamin A, vitamin D, vitamin E, vitamin C, folic acid, thiamine, riboflavin, niacin, vitamin B-6, vitamin B-12, biotin, and pantothenic acid. In choosing a vitamin, one of ordinary skill in the art recognizes that suitable salts and/or trituration solutions may be useful in the formulation in, for example, considering solubility. Non-limiting examples include tocopheryl acetate, vitamin A palmitate, cholecalcifero, phytonadione (K-1), choline bitartrate, thiamine hydrochloride (B-1), biotin trituration, 1%, niacinamide, calcium pantothenate, pyridoxine hydrochloride (B-6), folic acid, and sodium ascorbate.

Minerals are known in the art and include, but are not limited to, calcium, phosphorus, iodine, iron, magnesium, copper, zinc, sodium, manganese, potassium, chloride, potassium citrate, calcium gluconate, calcium glycerophosphate, magnesium chloride, magnesium oxide, copper gluconate, ferrous gluconate, zinc gluconate, manganese gluconate, and potassium iodide. In addition, taurine could be provided in weight percentages between 0.025 and 0.5. Similarly, because taurine is an essential amino acid for cats, addition of proper amounts of taurine are contemplated.

To select a specific mineral compound to be used in the diet requires consideration of that compound's chemical nature regarding compatibility with the shelf storage. In the prior art, a high proportion of gluconate salts, which are more expensive, represent the majority of mineral salts utilized because of their compatibility during processing (i.e., sterilization). However, the composition of the present invention does not require post-formulation processing and does not suffer from such restrictions on the selection of ingredients.

The inventive composition is provided as an aqueous solution, a liquid concentrate, or a colloidal suspension. The composition is characterized by having a physical consistency of a gel, a sol-gel, a gravy, or a syrup, and such a consistency is useful for the adsorbing on the outer surface of a pet food or to be mixed in a drink.

It is preferred that the inventive composition have a pH in the range of 4.0 to 8.0, or more preferably, a pH in the range of about 5.0 to about 6.5, and most preferably, a pH in the range of about 5.5 to about 6.5. A pH above about 8.0 is undesirable due to the bitter taste of alkaline products and thus, lacks palatability.

It is contemplated that other components such as suitable flavoring agents can also be used. If the inventive composition is to be provided for pet use, suitable flavorings can include beef, fish, veal, lamb, chicken, pork, cheese and the like. If the inventive composition is to be provided for human use, suitable flavorings can include cola, lemon, lime, lemon-lime, cherry, punch, orange, grape, root beer, strawberry and the like. These can be included by means of an artificial or natural flavor system. The flavoring agents are generally present in amounts of at least 0.02% by weight or above on a weight basis of the liquid beverage and can be varied to suite individual taste. For example, U.S. Pat. No. 3,773,930, issued Nov. 20, 1973 to K. Mohammed, et al, addresses improvement of flavor by incorporating pectin and various fruit flavorings. Coloring agents can also be incorporated into the compositions of the invention, if desired. The type of coloring agent used is not critical as long as it is not toxic and is approved for food use.

It is noted that certain flavor systems contain ingredients harmful to the stability of proteins that are exacerbated under sterilization conditions. For instance, some flavoring systems contain gum arabic which has been found to react immediately with protein after pasteurizing or within 24 hours in a cold pack beverage causing precipitation of the protein. Although avoiding use of materials that cause undesirable instability problems under sterilization conditions is not relevant to the present invention, a skilled artisan recognizes that shelf life may be adversely effected and the use of such materials should be considered. For example, storage capabilities of natural citrus juice drinks are not particularly long and, consequently, various preservatives must be incorporated therein.

Preservatives such as sodium benzoate and/or potassium sorbate can also be used if an ingredient is added that requires such. Levels of from about 0.01 to about 1% by weight of the composition are generally preferred. Sequestering agents such as ethylenediamine-tetraacetic acid and its salts such as the sodium, calcium salts may also be used for maintaining flavor and color. Generally, preferred amounts are from about 5 to about 500 parts per million, depending on the water supply used, the hardness of the water, and the metal content. Usually from about 30 to about 50 ppm. is adequate in good potable water.

It is an object of the present invention to provide a process for producing a ready-to-use composition for supplementing the nutritional content of a pet food comprising the step of mixing, on a dry matter basis, from about 15 to about 80% by weight of a protein component, from about 20 to about 85% by weight of a humectant, and from about 1 to about 50% by weight of a lipid component to form the composition, wherein the composition is microbiologically stable.

The composition is contemplated as useful for adsorbing on the outer surface of the pet food, such as a food topper. Further, the composition may further comprise from about 0.1 to about 49% by weight of a functional ingredient. Because the composition is microbiologically stable due to the synergy between the protein component and the humectant, the functional ingredient can be, but is not required to be, heat labile.

In other embodiments, the process further comprises adding a vitamin or a mineral. This is desirable if the inventive composition is to be used as a complete dietary system, however, this is not necessary in contemplating the addition of vitamins and/or minerals.

It is preferred that the protein component comprise hydrolyzed proteins, which is obtained from an animal or a plant source. In preferred embodiments, the protein component comprises gelatin. In this case, the gelatin is in either a powder form or a ready-to-use form (i.e., 50% aqueous solution). To determine which form of a protein component to use, the pH of the composition should be considered.

To afford the required microbiological stability and provide a carbohydrate component, the humectant comprises a sugar, a polyhydroxyl alcohol or a mixture of a sugar and a polyhydroxyl alcohol in a ratio of about 1:1, in a ratio of about 1:2, or in a ratio of about 2:3.

For palatability, the lipid component is a fatty acid, such as stearic acid, or a derivative thereof, such as a glycerol, an acyl, or an alkyl ester. In other specific embodiments, the lipid component is provided as a vegetable oil including coconut oil, corn oil, cotton seed oil, olive oil, safflower oil, sunflower oil, soybean oil, or an equivalent oil. In certain embodiments, the lipid component is added after the protein component and the humectant are mixed, although this is not necessary provided the composition is in solution. A skilled artisan is aware of techniques to use to determine solubility, which can be as simple as the absence of undissolved particulates.

In certain embodiments, the composition is an aqueous solution, a liquid concentrate, or a colloidal suspension. In other specific embodiments, the composition is characterized by having a physical consistency of a gel, a sol-gel, a gravy, or a syrup. Prior art compositions are typically provided in a foil packet which must be mixed with water just prior to use. Aside from the obvious inconvenience of having to mix a dry powder prior to use, it can be appreciated that the very act of mixing raises possibilities of contamination.

The mixtures of the present invention can be prepared by conventional mixing and blending techniques utilizing standard equipment. Components are milled to a suitable size and then mixed and blended in required amounts to form the mixtures which can be reconstituted with water or other fluid as desired. Separate ingredients can be mixed into liquid systems to facilitate dissolving.

Feeding companion animals, such as a dog or a cat, the pet food of the subject invention will enhance the nutritional intake of a domestic animal by providing a nutritional supplement having the nutrients desired to improve the health of the animal. For example, the inventive composition can be poured over a pet food kibble in which it would be adsorbed on the outer surface of the pet food product before the animal consumes the food. It is contemplated that the inventive composition can also be used as a delivery system for various nutrients, functional ingredients, pharmaceuticals and/or medicaments, especially those that are sensitive to heat. Thus, it is an object of the present invention that the inventive composition comprise a number of nutrients and/or functional ingredients, pharmaceuticals and medicaments such as including but not limited to sunflower or safflower oil, linoleic acid, ginger root extract, ginkgo extract; anti-inflammatory agents; breath improvement agents such as rosemary, clove and parsley seed oils; antioxidants such as vitamin E, the carotenoids (i.e., β-carotene, lycopene) and glucosamine sulfate; antibiotics; probiotics; and/or a vitamin, a mineral or combinations thereof. The inventive composition can also include ingredients that will improve or alleviate genetic conditions found in certain pet animals.

The nutrients are delivered to the animal by feeding the animal a pet food that is supplemented with the inventive ready-to-use composition comprising, on a dry matter basis, from about 15 to about 80% by weight of a protein component, from about 20 to about 85% by weight of a humectant, from about 1 to about 50% by weight of a lipid component, to form a nutritional delivery composition, wherein the composition is microbiologically stable. As a consequence of feeding the companion animal a food supplemented with the inventive composition, the animal is healthier.

In further embodiments, the composition further comprises from about 0.1 to 49% of an essential nutrient, and one of ordinary skill in the art understands that the essential nutrient comprises any compound that is required by the body and must be obtained through dietary intake.

In yet another further embodiments, the composition further comprises about 0.1 to about 49% by weight of a functional ingredient, and the functional ingredient can be heat labile.

In specific embodiments, the protein component comprises hydrolyzed protein, such as gelatin. In other specific embodiments, the humectant comprises a mixture of a sugar and a polyhydroxyl alcohol. In an additional specific embodiment, the composition has a pH in the range of from about 5.5 to about 6.5.

This invention also concerns a liquid composition so formulated that it provides useful nourishment for companion animals. In one embodiment, the companion animal has a compromised digestive function and/or malabsorption. This completely digestible composition comprises a protein component that is mixed with the humectant comprising dextrose or an equivalent sugar such as invert sugar or modified corn starch to provide microbiological stability; and a lipid component, preferably of a vegetable oil to provide palatability. The composition further comprises nutritionally significant amounts of essential vitamins and minerals and/or functional ingredients.

The instant invention is directed to a liquid ready-to-use medical food, for both humans and companion animals, comprising a protein, lipid, and carbohydrate components; as well as all the vitamins and minerals considered to be essential in a daily diet. It is intended that this composition may be used for enteral feeding, either orally or by intubation, for patients suffering from malnourishment and conditions associated therewith as well as for maintenance of animals with compromised digestive and/or absorptive function which can arise from a variety of causes. It is recognized in the art that the term “elemental diet” as applied to these liquid diet compositions generally refers to an enterically administered liquid diet which provides the patient's basic nutritional requirements in an elemental, easily digestible source.

The protein source in the inventive diet for medical use is generally a protein hydrolysate or the individual amino acids in purified form or a mixture of these. Carbohydrates, the main caloric source ingredient in these diets, usually comprise sucrose and/or glucose or small polymers of glucose. The percentage of calories supplied as fat is usually limited, and in some diets the major portion of the administered fat is in the form of essential fatty acids. Vitamins, electrolytes and trace elements are also available in these elemental diets to meet desired nutritional requirements. The methods and procedures for administration of medical foods are well known to those practitioners skilled in the pertinent art. It is to be understood that such feeding is generally done under direction of appropriately trained medical personnel.

Therefore, it is another object of the present invention to provide a ready-to-use composition for delivering a pharmaceutical or a medicament comprising on a dry matter basis, from about 15 to about 80% by weight of a protein component, from about 20 to about 85% by weight of a humectant, from about 1 to about 50% by weight of a lipid component, and a therapeutically effective amount of the pharmaceutical or the medicament, wherein the composition is microbiologically stable.

It is important that the pharmaceutical or the medicament is water soluble, which includes the ingredient being pre-solubilized in a suspension, or a colloidal suspension such as an emulsion or a liposomal complex. In specific embodiments, the pharmaceutical is an antibiotic and probiotic.

In certain embodiments, the protein component comprises hydrolyzed proteins that are from an animal source or a plant source, preferably the protein component comprises gelatin.

In specific embodiments, the humectant comprises a sugar, a polyhydroxyl alcohol or a mixture of a sugar and a polyhydroxyl alcohol. In specific embodiments, the mixture comprises the sugar and the polyhydroxyl alcohol in a ratio of about 1:1, a ratio of about 1:2, or a ratio of about 2:3.

In certain embodiments, the lipid component is a fatty acid or a derivative thereof. The lipid component is, in a specific embodiment, provided as a vegetable oil. The inventive composition is an aqueous solution, a liquid concentrate, or a colloidal suspension and is characterized by having a physical consistency of a gel, a sol-gel, a gravy, or a syrup.

In preferred specific embodiments, the composition has a pH in the range of about 4.0 to about 8.0, or more preferably, a pH in the range of about 5.0 to about 6.5, or most preferably, a pH in the range of about 5.5 to about 6.5.

A further object of the present invention is a process for delivering medicine to an animal comprising the steps of mixing, on a dry matter basis, about 15 to about 80% by weight of a protein component, about 20 to about 85% by weight of a humectant, about 1 to about 50% by weight of a lipid component, to form a delivery composition, wherein the composition is microbiologically stable; adding a therapeutically effective amount of a pharmaceutical in a pharmaceutically acceptable diluent to form a medicinal delivery composition; and feeding to the animal the medicinal delivery composition.

In specific embodiments, the pharmaceutical is an antibiotic or probiotic. In other specific embodiments, the animal is fed orally or enterally.

In certain embodiments, the protein component comprises gelatin, and the humectant comprises a mixture of a sugar and a polyhydroxyl alcohol. In specific embodiments, the composition has a pH in the range of about 5.5 to about 6.5.

As is known to one skilled in the art, there are a variety of commonly known pet food products. In the area of cat and dog food, there is wet pet food, semi-moist pet food, dry pet food and pet treats and snacks. Pet treats and snacks can typically be semi-moist chewable treats or snacks; dry treats or snacks in any number of forms; chewable bones; baked, extruded or stamped treats; confection treats/snacks; or other kinds of treats as is known to one skilled in the art. It is contemplated that the dietary supplement of the present invention is applied to the outer surface of the pet food if the pet food is in a solid or semi-solid form.

In certain embodiments, the inventive food is a microbiologically stable carrier comprised of a functional ingredient, such as a ginger root extract or an omega-3 fatty acid. In particular embodiments, the inventive food comprises functional ingredients that are heat labile, which is a particular advantage of the present invention. In specific embodiments, the composition is then adsorbed on a commercial pet food product and fed to the animal. In other specific embodiments, the composition is the adsorbed on a food suitable for human consumption and fed to the person.

In certain embodiments, the ready-to-use composition is used for supplementing the nutritional content of a pet food and is prepared by a process comprising adding from about 15 to about 80% by weight of a protein component to an equal amount of water to form a solution; mixing into said solution from about 20 to about 85% by weight of a carbohydrate component to form a mixture; and combining from about 1 to about 50% by weight of a lipid component to the mixture to produce the composition. A functional ingredient and/or an essential nutrient is then added to the composition mixture in about 0.1 to about 49% by weight.

The procedure for preparing the pet food composition of the present invention is variable. For example, in certain embodiments, the preparation temperature does not exceed about 70 degrees C. and some or all of the components are added during preparation. In such embodiments, the functional ingredient is added directly to the composition during preparation. However, the preparation temperature may exceed 70 degrees C., and, in such cases, the preparation may, optionally, be cooled to below 70 degrees C. prior to the addition of the functional ingredient.

The composition of the present invention is microbiologically stable. Therefore, it is contemplated that the composition functions as a preservative system to be used in the preparation of a pet food. In one specific embodiment, the inventive composition is included as an ingredient in a pet food product to replace a conventional preservative such as polyethylene glycol, thereby functioning as a preservative. In another specific embodiment, the inventive composition is included as an ingredient in an intermediate pet food product, which is any pet food product that is incompletely formulated and/or processed, as a preservative. The incorporation of the inventive composition is considered for purposes of initiating and/or maintaining microbiological stability of the pet food. In alternative embodiments, the inventive composition functions not only a preservative but also as a dietary delivery system. This is particularly advantageous for the preparation of nutritionally incomplete pet food products.

A skilled artisan is aware that a pet food product further comprising vitamins, trace minerals and flavorings, is within the scope of the present invention, and is preferred to provide the animal with an entire daily diet to manage and promote healthy living.

The process of feeding the companion animal the inventive pet food product will promote healthy living in the companion animals. Thus, as described herein, the present invention provides a comprehensive approach to enhance the nutrition intake of a domestic animal. The invention is directed to a microbiologically stable composition that is useful as a delivery means, in particular for nutrients, that also provides nutritional supplementation to the animal.

### EXAMPLE 1

### The Delivery Composition

In preparing the inventive composition, mixing procedures are optimized to establish a proper mixing procedure for the humectant and the protein component. Representative humectants were investigated for their influence on the activity of water in the finished composition.

In one embodiment, a commercial mixer such as a Hobart mixer was used to prepare 2 kg of a protein component comprising an aqueous gelatin solution. In a preferred embodiment, the gelatin solution is 50%. To prepare the gelatin solution, gelatin was dissolved in cold water (C-batch) and in water at the temperature of about 45° C. (H-batch).

The H-batch prepared was found to require significantly reduced (8 minutes as compared to 15 minutes) mixing times to dissolve the gelatin as compared to the C-batch. Furthermore, the amount of foam produced in the H-batch was less than the C-batch. Water activity and pH of gelatin solutions prepared from powder and ready to use gelatin (i.e., Polypro 5000) were measured. The ready to use gelatin exhibited a lower pH (pH of 3.9) and slightly lower water activity (less than 1) as compared to the gelatin solution prepared from powder (pH of 5.9).

Approximately 80 g of each gelatin solution (about 40% on a dry matter basis) was mixed with various humectants in varied compositions (about 50% on a dry matter basis) including glycerin, sugar, invert sugar, and glucose. These samples provided a base preparation that further comprised about 10% of a lipid component, which in one embodiment comprised sunflower oil.

The water activity, pH and temperature of the each base sample was recorded. It is recognized that a low water activity yields a higher pH. The measured pH ranges included from about 5.2 to 5.7 During the preparation of the gelatin solution with cold water, 15 min mixing time was required to fully dissolve the gelatin powder. Long mixing time caused extensive aeration and generation of a thick foam. In this event, the solution had to be set aside for more than 1 hour in order to partially return to a liquid form. Therefore, it is preferred to use hot water to prepare the hydrolyzed protein solution, but this is not necessary.

Based on the results above, sugar proved to be the most effective humectant by providing a base with the relatively lowest water activity. Further, previous samples of bases were prepared with ready to use gelatin solution, which provided a finished base with pH of 4.2.

### EXAMPLE 2

### The Process of Preparing the Delivery Composition

One object of the present invention is a process of preparing the inventive composition. To this end, a validating pilot plant run was performed.

In one embodiment, the composition comprised about 40% of a gelatin solution, about 50% of a humectant that comprised a blend of sugars and a polyhydroxyl alcohol and about 10% of a lipid component. The batches were prepared in three steps. First, the mixer was filled with 151 kg of hot water at the temperature of 50 to 60° C., and the agitators were set at 9 RPM for outer, and 20 RPM for the center agitator. The Polypro 5000 was added to the mixer and the jacked was steam heated until the temperature reached a temperature in the range of about 65 to about 70° C. Second, to prevent premature browning , the heating of the mixer bowl was decreased and the humectant mixture was added to the mixer. The temperature was in the range of about 40 to about 48° C. Third, the steam valve leading to the mixer bowl jacket was opened, and in the last step, the lipid component was added. The base was constantly agitated and heated until the temperature of the base reached the required 90° C.

A skilled artisan recognizes that the temperature of 90° C. is maintained for a maximum of 10 min before the sugar caramelization affects the color of the solution.

Additional testing included the addition of an antifoam agent (Antifoam 1520), which was added at the beginning of step1 and the speed of the agitators was increased to 11 RPM for outer, and 26 RPM for inner agitator. In another embodiment, the lipid component is mixed with a processing aid, such as lecithin, to improve the emulsification. The amount of foam developed during mixing was significantly reduced to a layer of 1.5-2 inches, and avoided by addition of the antifoaming agent in which case the mixing speed was increased.

Thus, in one embodiment of the present process, a combination of two agitators incorporated in one mixer is contemplated, in particular in the event that a maximum mixing action without incorporating air is desired. Also contemplated, is reducing preparation time by maintaining a temperature above ambient temperature to prepare the protein component, if the protein component is dissolved in a first solution.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification.

### REFERENCES

The following references, to the extent that they provide exemplary procedural or other details supplementary to those set forth herein, are specifically incorporated herein by reference:

### Patents

U.S. Pat. No. 4,497,800 to Larson et al.

U.S. Pat. No. 4,414,238 to Schmidl

U.S. Pat. No. 4,070,488 to Davis

U.S. Pat. No. 5,017,389 to Green

U.S. Pat. No. 3,773,930 to Mohammed et al.

U.S. Pat. No. 6,071,544

U.S. Pat. No. 5,141,755

U.S. Pat. No. 4,892,748

U.S. Pat. No. 5,962,043

U.S. Pat. No. 6,204,291

U.S. Pat. No. 3,810,994

U.S. Pat. No. 3,830,931

U.S. Pat. No. 3,968,241

U.S. Pat. No. 4,255,449

EP 646325A1

Yalkowsky, S. H., 1970

We claim:

1. A ready-to-use composition for supplementing the nutritional content of pet food comprising, on a dry matter basis, from about 15 to about 80% by weight of a protein component, from about 20 to about 85% by weight of a humectant, and from about 1 to about 50% by weight of a lipid component, wherein the composition is microbiologically stable.

2. The composition of claim 1, further comprising from about 0.1 to about 49% by weight of a functional ingredient.

3. The composition of claim 2, wherein the functional ingredient in heat labile.

4. The composition of claim 3, wherein the heat labile functional ingredient comprises an essential oil, a volatile molecule having a fragrance, a molecule that is readily oxidized, denatured or inactivated, an enzyme, an antibiotic or a probiotic.

5. The composition of claim 1, wherein said protein component comprises hydrolyzed proteins.

6. The composition of claim 5, wherein said hydrolyzed protein is from an animal or a plant source.

7. The process of claim 6, wherein said animal source comprises beef, swine, sheep, fish, or poultry.

8. The process of claim 6, wherein said plant source comprises wheat, alfalfa or legume.

9. The composition of claim 1, wherein said protein component comprises gelatin.

10. The composition of claim 1, wherein said humectant comprises a sugar.

11. The composition of claim 1, wherein said humectant comprises a polyhydroxyl alcohol.

12. The composition of claim 1, wherein the humectant comprises a mixture of a sugar and a polyhydroxyl alcohol.

13. The composition of claim 12, wherein the mixture comprises the sugar and the polyhydroxyl alcohol in a ratio of about 1:1.

14. The composition of claim 12, wherein the mixture comprises the sugar and the polyhydroxyl alcohol in a ratio of about 1:2.

15. The composition of claim 12, wherein the mixture comprises the sugar and the polyhydroxyl alcohol in a ratio of about 2:3.

16. The composition of claim 1, wherein the lipid component is a fatty acid or a derivative thereof.

17. The composition of claim 16, wherein the fatty acid comprises lauric acid, myristic acid, palmitic acid, stearic acid, arachidic acid, behenic acid, lignoceric acid, palmitoleic acid, oleic acid, linoleic acid, alpha-linoleic acid, gamma-linoleic acid, arahidonic acid, nervonic acid, eicosapentanoic acid, an omega-3 fatty acid or derivatives thereof.

18. The composition of claim 1, wherein the lipid component is provided as a vegetable oil.

19. The composition of claim 18, wherein the vegetable oil comprises coconut oil, corn oil, cotton seed oil, olive oil, safflower oil, sunflower oil, soybean oil, or an equivalent oil.

20. The composition of claim 1, further comprising a vitamin.

21. The composition of claim 1, further comprising a mineral.

22. The composition of claim 1, wherein said composition is an aqueous solution, a liquid concentrate, or a colloidal suspension.

23. The composition of claim 1, wherein the composition is characterized by having a physical consistency of a gel, a sol-gel, a gravy, or a syrup.

24. The composition of claim 1, wherein said composition has a pH in the range of about 4.0 to about 8.0.

25. The composition of claim 1, wherein said composition has a pH in the range of about 5.0 to about 6.5.

26. The composition of claim 1, wherein said composition has a pH in the range of about 5.5 to about 6.5.

27. A ready-to-use composition for delivering a pharmaceutical or a medicament comprising on a dry matter basis, from about 15 to about 80% by weight of a protein component, from about 20 to about 85% by weight of a humectant, from about 1 to about 50% by weight of a lipid component, and a therapeutically effective amount of the pharmaceutical or the medicament, wherein the composition is microbiologically stable.

28. The composition of claim 27, wherein the pharmaceutical is water soluble.

29. The composition of claim 27, wherein the pharmaceutical is an antibiotic.

30. The composition of claim 27, wherein the medicament is water soluble.

31. The composition of claim 27, wherein said protein component comprises hydrolyzed proteins.

32. The composition of claim 31, wherein said hydrolyzed protein is from an animal or a plant source.

33. The process of claim 32, wherein said animal source comprises beef, swine, sheep, fish, or poultry.

34. The process of claim 32, wherein said plant source comprises wheat, alfalfa or legume.

35. The composition of claim 27, wherein said protein component comprises gelatin.

36. The composition of claim 27, wherein said humectant comprises a sugar.

37. The composition of claim 27, wherein said humectant comprises a polyhydroxyl alcohol.

38. The composition of claim 27, wherein the humectant comprises a mixture of a sugar and a polyhydroxyl alcohol.

39. The composition of claim 38, wherein the mixture comprises the sugar and the polyhydroxyl alcohol in a ratio of about 1:1.

40. The composition of claim 38, wherein the mixture comprises the sugar and the polyhydroxyl alcohol in a ratio of about 1:2.

41. The composition of claim 38, wherein the mixture comprises the sugar and the polyhydroxyl alcohol in a ratio of about 2:3.

42. The composition of claim 27, wherein the lipid component is a fatty acid or a derivative thereof.

43. The composition of claim 42, wherein the fatty acid comprises lauric acid, myristic acid, palmitic acid, stearic acid, arachidic acid, behenic acid, lignoceric acid, palmitoleic acid, oleic acid, linoleic acid, alpha-linoleic acid, gamma-linoleic acid, arahidonic acid, nervonic acid, eicosapentanoic acid or derivatives thereof.

44. The composition of claim 27, wherein the lipid component is provided as a vegetable oil.

45. The composition of claim 44, wherein the vegetable oil is coconut oil, corn oil, cotton seed oil, olive oil, safflower oil, sunflower oil, soybean oil, or an equivalent oil.

46. The composition of claim 27, wherein said composition is an aqueous solution, a liquid concentrate, or a colloidal suspension.

47. The composition of claim 27, wherein said composition is characterized by having a physical consistency of a gel, a sol-gel, a gravy, or a syrup.

48. The composition of claim 27, wherein said composition has a pH in the range of about 4.0 to about 8.0.

49. The composition of claim 27, wherein said composition has a pH in the range of about 5.0 to about 6.5.

50. The composition of claim 27, wherein said composition has a pH in the range of about 5.5 to about 6.5.

51. A process for producing a ready-to-use composition for supplementing the nutritional content of a pet food comprising the step of mixing, on a dry matter basis, from about 15 to about 80% by weight of a protein component, from about 20 to about 85% by weight of a humectant, and from about 1 to about 50% by weight of a lipid component to form the composition, wherein the composition is microbiologically stable.

52. The process of claim 51, further comprising adsorbing the composition on the outer surface of the pet food.

53. The process of claim 51, further comprising adding from about 0.1 to about 49% by weight of a functional ingredient to the composition.

54. The process of claim 53, wherein the functional ingredient is heat labile.

55. The process of claim 51, further comprising adding a vitamin to the composition.

56. The process of claim 51, further comprising adding a mineral to the composition.

57. The process of claim 51, wherein said protein component comprises hydrolyzed proteins.

58. The process of claim 57, wherein said hydrolyzed protein is from an animal or a plant source.

59. The process of claim 58, wherein said animal source comprises beef, swine, sheep, fish, or poultry.

60. The process of claim 58, wherein said plant source comprises a wheat, an alfalfa or a legume source.

61. The process of claim 51, wherein said protein component comprises gelatin.

62. The process of claim 51, wherein said humectant comprises a sugar.

63. The process of claim 51, wherein said humectant comprises a polyhydroxyl alcohol.

64. The process of claim 51, wherein the humectant comprises a mixture of a sugar and a polyhydroxyl alcohol.

65. The process of claim 64, wherein the mixture comprises the sugar and the polyhydroxyl alcohol in a ratio of about 1:1.

66. The process of claim 64, wherein the mixture comprises the sugar and the polyhydroxyl alcohol in a ratio of about 1:2.

67. The process of claim 66, wherein the mixture comprises the sugar and the polyhydroxyl alcohol in a ratio of about 2:3.

68. The process of claim 51, wherein the lipid component is a fatty acid or a derivative thereof.

69. The composition of claim 68, wherein the fatty acid comprises lauric acid, myristic acid, palmitic acid, stearic acid, arachidic acid, behenic acid, lignoceric acid, palmitoleic acid, oleic acid, linoleic acid, alpha-linoleic acid, gamma-linoleic acid, arahidonic acid, nervonic acid, eicosapentanoic acid or derivatives thereof.

70. The process of claim 51, wherein the lipid component is provided as a vegetable oil.

71. The process of claim 70, wherein the vegetable oil is coconut oil, corn oil, cotton seed oil, olive oil, safflower oil, sunflower oil, soybean oil, or an equivalent oil.

72. The process of claim 5 1, wherein said composition is an aqueous solution, a liquid concentrate, or a colloidal suspension.

73. The process of claim 51, wherein the composition is characterized by having a physical consistency of a gel, a sol-gel, a gravy, or a syrup.

74. The process of claim 51, wherein said composition has a pH in the range of about 4.0 to about 8.0.

75. The process of claim 51, wherein said composition has a pH in the range of about 5.0 to about 6.5.

76. The process of claim 51, wherein said composition has a pH in the range of about 5.5 to about 6.5.

77. A process for producing a ready-to-use composition for supplementing the nutritional content of a pet food, the process comprising the steps of:
adding from about 15 to about 80% by weight of a protein component to an equal amount of water to form a solution;
mixing into said solution from about 20 to about 85% by weight of a carbohydrate component to form a mixture; and
combining from about 1 to about 50% by weight of a lipid component to the mixture to produce the composition.

78. The process of claim 77, further comprising the step of adding from about 0.1 to about 49% by weight of a functional ingredient to the mixture or the composition.

79. The process of claim 78, wherein the functional ingredient is heat labile.

80. The process of claim 77, further comprising adding a vitamin to the composition.

81. The process of claim 77, further comprising adding a mineral to the composition.

82. The process of claim 77, wherein said protein component comprises hydrolyzed proteins.

83. The process of claim 82, wherein said hydrolyzed protein is from an animal or a plant source.

84. The process of claim 83, wherein said animal source comprises beef, swine, sheep, fish, or poultry.

85. The process of claim 83, wherein said plant source comprises wheat, alfalfa or legume.

86. The process of claim 77, wherein said protein component comprises gelatin.

87. The process of claim 77, wherein said humectant comprises a sugar.

88. The process of claim 77, wherein said humectant comprises a polyhydroxyl alcohol.

89. The process of claim 77, wherein the humectant comprises a mixture of a sugar and a polyhydroxyl alcohol.

90. The process of claim 89, wherein the mixture comprises the sugar and the polyhydroxyl alcohol in a ratio of about 1:1.

91. The process of claim 89, wherein the mixture comprises the sugar and the polyhydroxyl alcohol in a ratio of about 1:2.

92. The process of claim 89, wherein the mixture comprises the sugar and the polyhydroxyl alcohol in a ratio of about 2:3.

93. The process of claim 77, wherein the lipid component is a fatty acid or a derivative thereof.

94. The composition of claim 93, wherein the fatty acid comprises lauric acid, myristic acid, palmitic acid, stearic acid, arachidic acid, behenic acid, lignoceric acid, palmitoleic acid, oleic acid, linoleic acid, alpha-linoleic acid, gamma-linoleic acid, arahidonic acid, nervonic acid, eicosapentanoic acid or derivatives thereof.

95. The process of claim 77, wherein said composition is an aqueous solution, a liquid concentrate, or a colloidal suspension.

96. The process of claim 77, wherein the composition is characterized by having a physical consistency of a gel, a sol-gel, a gravy, or a syrup.

97. The process of claim 77, wherein said composition has a pH in the range of about 4.0 to about 8.0.

98. The process of claim 77, wherein said composition has a pH in the range of about 5.5 to about 6.5

99. A process for delivering a nutrient to an animal comprising the step of feeding to an animal a ready-to-use composition comprising, on a dry matter basis, from about 15 to about 80% by weight of a protein component, from about 20 to about 85% by weight of a humectant, from about 1 to about 50% by weight of a lipid component, and from about 0.1 to 49% of an essential nutrient to form a nutritional delivery composition, wherein the composition is microbiologically stable.

100. A process for delivering medicine to an animal comprising the steps of:
mixing, on a dry matter basis, about 15 to about 80% by weight of a protein component, about 20 to about 85% by weight of a humectant, about 1 to about 50% by weight of a lipid component, to form a delivery composition, wherein the composition is microbiologically stable;
adding a therapeutically effective amount of a pharmaceutical in a pharmaceutically acceptable diluent to form a medicinal delivery composition; and
feeding to said animal the medicinal delivery composition.

101. The process of claim 100, wherein the pharmaceutical is an antibiotic.

102. The process of claim 100, wherein feeding comprises oral feeding or enteral feeding.

103. The process of claim 100, wherein the protein component comprises gelatin.

104. The process of claim 100, wherein the humectant comprises a mixture of a sugar and a polyhydroxyl alcohol.

105. The process of claim 100, wherein the composition has a pH in the range of about 5.5 to about 6.5.

106. A process of promoting nutrition in a companion animal comprising the step of feeding to said animal a pet food having an outer layer comprised of from about 15 to about 80% by weight of a protein component, from about 20 to about 85% by weight of a humectant, and from about 1 to about 50% by weight of a lipid component, wherein the composition is microbiologically stable.

107. The process of claim 106, wherein the composition further comprises about 0.1 to about 49% by weight of a functional ingredient.

108. The process of claim 107, wherein the functional ingredient is heat labile.

109. The process of claim 106, wherein the protein component comprises hydrolyzed protein.

110. The process of claim 106, wherein the protein component comprises gelatin.

111. The process of claim 106, wherein the humectant comprises a mixture of a sugar and a polyhydroxyl alcohol.

112. The process of claim 106, wherein the composition has a pH in the range of about 5.5 to about 6.5.
